(19) United States
(12) Patent Application Publication
(10) Pub. No.: US 2022/0313569 A1
(43) Pub. Date: Oct. 6, 2022

(54) ANTIPERSPIRANT ACTIVE EMULSION

(71) Applicant: BEIERSDORF AG, Hamburg (DE)

(72) Inventors: Melanie VOGT, Suelfeld (DE); Ilka OELRICHS, Tornesch (DE)

(21) Appl. No.: 17/753,438

(22) PCT Filed: Sep. 4, 2019
(86) PCT No.: PCT/EP2019/073544

§ 371 Date: Mar. 3, 2022

(51) Int. Cl. A61K 8/06; A61K 8/26; A61K 8/41; A61Q 15/00; A61K 8/81; A61K 8/92

(52) U.S. Cl. CPC A61K 8/062; A61K 8/26; A61K 8/416; A61Q 15/00; A61K 8/8152; A61K 8/922; A61K 2800/21; A61K 2800/805

(57) ABSTRACT

The present invention belongs to the cosmetic field and relates to a cosmetic oil in water emulsion in stick form

The present invention belongs to the cosmetic field and relates to an emulsion which forms an antiperspirant stick.

Solid cosmetic antiperspirant sticks which are in a form of an oil in water emulsion are commonly known by the person skilled in art.

E.g. the document WO 2006/012971 A1 discloses low-residue antiperspirant sticks on the basis of an oil in water dispersion. The stick compositions disclosed comprise an oil phase, emulsifier, an aqueous phase and aluminum components, which act as antiperspirant active material.

Additionally WO2017/162443 A1 discloses cosmetic antiperspirant sticks in form of an oil in water dispersion with improved from stability.

Furthermore, WO 2012/080017 A2 discloses in the examples 1.1 to 1.7 various antiperspirant sticks on the basis of an oil in water dispersion.

The pure fact that the person skilled in art knows a few antiperspirant sticks in form of an oil in water dispersion shall not obscure the fact that these prior art sticks suffer a number of disadvantages. One particular disadvantage is directed to the mass production of such emulsion sticks. Conventionally, the sticks are prepared by melting all ingredients of the oil phase and mixing the ingredients of the aqueous phase. Both phases are mixed at elevated temperatures. Furthermore, in order to form an emulsion the mixture is homogenized and afterwards stored at elevated temperatures, e.g. 70° C., until the obtained bulk is filled into a mold for cooling and forming the stick. The mold is usually the final packaging of the product.

Specific problems arise from the fact that it often occurs that the homogenized bulk is stored for several hours before all of the bulk is filled into the packaging. Within that time it is often observed that agglomerated particles are formed within the bulk. These can also be described as lumps. Accordingly, it is often needed to filter the bulk before filling it into the packaging. As such measures increase the production costs due to the use and maintenance of the filters it is generally desirable to avoid the formation of agglomerated particles.

An analysis has indicated that such particles mainly contain aluminum compounds, oils and waxes. Consequently, by the formation of agglomerated particles the active content of the antiperspirant active aluminum compound is reduced. This may lead to a reduced antiperspirant efficiency in the final product. Or higher quantities of the antiperspirant active ingredient has to be included in the bulk which makes the product more expensive to produce.

Accordingly, it is the objective of the present invention to provide an emulsion which can be stored at elevated temperatures, in particular at 70° C., for a prolonged amount of time, e.g. 8 hours, which does not form agglomerated particles in the bulk. It was surprisingly found that these objectives can be addressed by the objects of the present invention.

The present invention is an antiperspirant oil in water emulsion comprising, calculated to the total weight of the emulsion,
a) At least one antiperspirant active aluminum compound;
b) Palmitamidopropyltrimonium chloride;
c) at least one lipid and/or wax component with a melting point of >50° C.;
d) at least one oil which is liquid at 20° C.;
e) at least one nonionic emulsifier with an HLB value of more than 7;
f) at least one nonionic emulsifier with an HLB value greater than 1.0 and less than or equal to 7.0; and
g) 30% to 50% by weight water.

All the weight percentages (% by weight) given below are based, unless otherwise stated, on the total weight of the emulsion of the invention. If ratios of certain components are disclosed in the following description, these ratios refer, unless otherwise stated, to weight ratios of the components.

Unless otherwise stated, all tests and measurements were performed under “normal conditions”. The term “normal conditions” refers to 20° C., 1013 hPa and a relative humidity of 50%.

The term “skin” refers solely to human skin.

It was surprisingly found that the emulsion of the present invention does not form clumps in the bulk when being stored at elevated temperatures for a prolonged amount of time, e.g. 4 hours, in particular 8 hours.

According to the present invention the emulsion comprises at least one antiperspirant active aluminum compound. According to the invention it is preferred if this aluminum compound is selected from the group consisting of aluminum chlorohydrate; aluminum sesquichlorohydrate; aluminum zirconium chlorohydrates, such as aluminum zirconium trichlorohydrate, aluminum zirconium tetrachlorohydrate, aluminum zirconium pentachlorohydrate, aluminum zirconium octachlorohydrate; and the aluminum zirconium chlorohydrate glycine complexes such as aluminum zirconium trichlorohydrex glycine, aluminum zirconium tetrachlorohydrex glycine, aluminum zirconium pentachlorohydrex glycine and aluminum zirconium octachlorohydrex glycine. It is in particular preferred if aluminum chlorohydrate and/or aluminum sesquichlorohydrate are contained as antiperspirant active aluminum compound.

Embodiments which are also preferred according to the invention are characterized in that only aluminum chlorohydrate and/or aluminum sesquichlorohydrate are contained as antiperspirant active aluminum compound.

The following description is valid for all embodiments of the invention.

It is preferred if the total quantity of the antiperspirant active aluminum compound is in the range from 5 to 30% by weight, more preferably 10 to 25% by weight and most preferably 12.5 to 22.5% by weight, calculated to the total weight of the emulsion.

It is particular preferred if aluminum chlorohydrate and/or aluminum sesquichlorohydrate are contained as antiperspirant active aluminum compound and the total quantity of aluminum chlorohydrate and/or aluminum sesquichlorohydrate is in the range from 5 to 30% by weight, more preferably 10 to 25% by weight and most preferably 12.5 to 22.5% by weight, calculated to the total weight of the emulsion.

It is of particular advantage if aluminum chlorohydrate is contained as antiperspirant active aluminum compound and the total quantity of aluminum chlorohydrate is in the range from 5 to 30% by weight, more preferably 10 to 25% by weight and most preferably 12.5 to 22.5% by weight, calculated to the total weight of the emulsion. Here, it is further preferred, if no further antiperspirant active aluminum compound is contained.

For the cases that the emulsion comprises aluminum chlorohydrate and aluminum sesquichlorohydrate it is further preferred, if the ratio by weight between the aluminum chlorohydrate and aluminum sesquichlorohydrate is preferably in the range from 2:1 to 50:1, more preferably from 3.5:1 to 30:1, still more preferably 4:1 to 24:1 and most preferable 5:1 to 20:1.

According to the invention the emulsion also comprises palmitamidopropyltrimonium chloride, whereby it is favored if the total quantity of palmitamidopropyltrimonium chloride is in the range from 0.1 to 5% by weight, more preferably 0.2 to 3% by weight, more preferably 0.3 to 2% by weight, more preferably 0.4 to 1.5% by weight and most preferably 0.5 to 1.2% by weight, calculated to the total weight of the emulsion.

According to the invention the emulsion comprises at least one lipid and/or wax component with a melting point of >50° C. Thereby, it is preferred if the total quantity of the lipid and/or wax components with a melting point of >50° C. is in the range from 8 to 18% by weight, more preferably 10 to 15% by weight calculated to the total weight of the emulsion.

Generally, waxes are of solid to brittle consistency, coarse to finely crystalline, transparent to opaque, but not glass-like, and melt above 50° C. without decomposition. Just a little above the melting point they are of low viscosity and exhibit a heavily temperature-dependent consistency and solubility.

According to the invention, preference is given, to natural vegetable waxes, e.g., candelilla wax, carnauba wax, japan wax, sugar cane wax, ouricoury wax, cork wax, sunflower wax, fruit waxes, such as orange waxes, lemon waxes, grapefruit wax, and animal waxes, e.g., beeswax, shellac wax and spermaceti. For the purposes of the invention, it may be particularly preferred to use hydrogenated or hardened waxes. Wax components which can be used are also chemically modified waxes, in particular, the hard waxes, such as, for example, montan ester waxes, hydrogenated jojoba waxes and sasol waxes. Synthetic waxes, which are likewise preferred according to the invention are polyalkylene waxes and polyethylene glycol waxes, C20-C40-dialkyl esters of dimer acids, C30-50-alkyl beeswax and alkyl and alkylaryl esters of dimer fatty acids.

A particularly preferred wax component is chosen from at least one ester of a saturated monohydric C16-C60-alcohol and a saturated C8-C36-monocarboxylic acid, as long as those esters have a melting point above 50° C. It is particularly preferred to choose the wax components from the group of esters of saturated, branched or unbranched alkanecarboxylic acids of chain length from 12 to 24 carbon atoms and the saturated, branched or unbranched alcohols of chain length from 16 to 50 carbon atoms which have a melting point of >50° C. It is most preferred if the emulsion comprises at least cetyl palmitate as wax and/or lipid component with a melting point of >50° C.

If an ester of saturated, branched or unbranched alkanecarboxylic acids of chain length from 12 to 24 carbon atoms and the saturated, branched or unbranched alcohols of chain length from 16 to 50 carbon atoms, which has a melting point of >50° C., is contained in the emulsion of the invention, it is further preferred if the total quantity of this ester is in the range from 3 to 9% by weight, more preferably 4.5 to 7.5% by weight an most preferably, 5 to 7% by weight, calculated to the total weight of the emulsion.

If cetyl palmitate is contained in the emulsion of the invention, it is further preferred if the total quantity of cetyl palmitate is in the range from 3 to 9% by weight, more preferably 4.5 to 7.5% by weight an most preferably, 5 to 7% by weight, calculated to the total weight of the emulsion.

Furthermore, it is preferred according to the invention if the emulsion also comprises C20-C40-alkyl stearate as wax component. If C20-C40-alkyl stearate is contained, it is further preferred if the total quantity of C20-C40-alkyl stearate is in the range from 0.2 to 3% by weight, more preferably 0.5 to 2% by weight and most preferably 0.75 to 1.5% by weight, calculated to the total weight of the emulsion.

Further preferred lipid and/or wax components are the triglycerides of saturated and optionally hydroxylated C12-30-fatty acids, such as hydrogenated triglyceride fats, in particular hydrogenated palm oil, hydrogenated coconut oil, hydrogenated castor oil, glyceryl tribehenate (tribehenin) and/or glyceryl tri-12-hydroxystearate, also synthetic complete esters of fatty acids and glycols or polyols having 2-6 carbon atoms so long as they have a melting point above 50° C., for example, preferably C18-C36 acid triglyceride (Syncrowax® HGL-C).

According to the invention, hydrogenated castor oil, obtainable, e.g., as the commercial product Cutina® HR, is a further particularly preferred wax component which has a melting point exceeding 50° C. In the case hydrogenated castor oil is contained in the emulsion of the invention, it is further preferred if the total quantity of hydrogenated castor oil in the emulsion is in the range from 3 to 9% by weight, more preferably 4.5 to 7.5% by weight an most preferably, 5 to 7% by weight, calculated to the total weight of the emulsion.

Further preferred lipid or wax components with a melting point of >50° C. are the saturated linear C14-C36-carboxylic acids, in particular myristic acid, palmitic acid, stearic acid and behenic acid, and mixtures of these compounds, e.g., Syncrowax® AW 1C (C18-C36-fatty acids) or Cutina® FS 45 (palmitic and stearic acid). According to the invention saturated linear C14-C36-carboxylic acids are not considered as emulsifier. In the case the emulsion comprises one or more saturated linear C14-C36-carboxylic acids, the total quantity of those carboxylic acid is preferably in the range from 1 to 8% by weight, more preferably 2 to 6% by weight and most preferably 3 to 5% by weight, calculated to the total weight of the emulsion.

A particular preferred embodiment of the invention is characterized in that the emulsion comprises the following lipid or wax components with a melting point of >50° C.:
an ester of saturated, branched or unbranched alkanecarboxylic acids of chain length from 12 to 24 carbon atoms and the saturated, branched or unbranched alcohols of chain length from 16 to 50 carbon atoms, which has a melting point of >50° C., preferably in a total quantity from 3 to 9% by weight, more preferably 4.5 to 7.5% by weight an most preferably, 5 to 7% by weight;
Hydrogenated castor oil, preferably in a total quantity from 3 to 9% by weight, more preferably 4.5 to 7.5% by weight an most preferably, 5 to 7% by weight; and
One or more saturated linear C14-C36-carboxylic acids, preferably in a total quantity from 1 to 8% by weight, more preferably 2 to 6% by weight and most preferably 3 to 5% by weight;

wherein the % by weight are calculated to the total weight of the emulsion.

Within this embodiment it is even more preferred, if the emulsion comprises the following lipid or wax components with a melting point of >50° C.:
Cetyl palmitate, preferably in a total quantity from 3 to 9% by weight, more preferably 4.5 to 7.5% by weight an most preferably, 5 to 7% by weight;
Hydrogenated castor oil, preferably in a total quantity from 3 to 9% by weight, more preferably 4.5 to 7.5% by weight an most preferably, 5 to 7% by weight; and
One or more saturated linear C14-C36-carboxylic acids, preferably in a total quantity from 1 to 8% by weight, more preferably 2 to 6% by weight and most preferably 3 to 5% by weight;

wherein the % by weight are calculated to the total weight of the emulsion.

Within this embodiment it is most preferred, if the emulsion comprises the following lipid or wax components with a melting point of >50° C.:
Cetyl palmitate, preferably in a total quantity from 3 to 9% by weight, more preferably 4.5 to 7.5% by weight an most preferably, 5 to 7% by weight;
Hydrogenated castor oil, preferably in a total quantity from 3 to 9% by weight, more preferably 4.5 to 7.5% by weight an most preferably, 5 to 7% by weight;
One or more saturated linear C14-C36-carboxylic acids, preferably in a total quantity from 1 to 8% by weight, more preferably 2 to 6% by weight and most preferably 3 to 5% by weight; and
C20-C40-alkyl stearate, preferably in the range from 0.2 to 3% by weight, more preferably 0.5 to 2% by weight and most preferably 0.75 to 1.5% by weight;

wherein the % by weight are calculated to the total weight of the emulsion.

Furthermore, the emulsion of the invention comprises at least one oil which is liquid at 20° C. The oil is preferably not a fragrance component or an essential oil. Preferred Oils are chosen from branched saturated or unsaturated fatty alcohols having 6-30 carbon atoms, which are liquid at 20° C. These alcohols are also often referred to as Guerbet alcohols since they are obtainable according to the Guerbet reaction.

Further oils d) preferred according to the invention are chosen from the triglycerides of linear or branched, saturated or unsaturated, optionally hydroxylated C8-30-fatty acids. The use of natural oils, e.g., soya oil, cotton seed oil, sunflower oil, palm oil, palm kernel oil, linseed oil, almond oil, castor oil, corn oil, olive oil, rapeseed oil, sesame oil, thistle oil, wheat germ oil, peach kernel oil and the liquid fractions of coconut oil and the like may be particularly suitable. Also suitable, however, are synthetic triglyceride oils, in particular capric/caprylic triglycerides.

Further oils d) preferred according to the invention are chosen from the dicarboxylic acid esters of linear or branched C2-C10-alkanols, in particular, diisopropyl adipate, di-n-butyl adipate, di(2-ethylhexyl) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate and di(2-hexyldecyl) succinate.

Further oils d) preferred according to the invention are chosen from the addition products of from 1 to 5 propylene oxide units onto mono- or polyhydric C8-22-alkanols, such as octanol, decanol, decanediol, lauryl alcohol, myristyl alcohol and stearyl alcohol, e.g., PPG-2 myristyl ether and PPG-3 myristyl ether.

Further oils d) are preferably chosen from the esters of the linear or branched saturated or unsaturated fatty alcohols having 2-30 carbon atoms with linear or branched saturated or unsaturated fatty acids having 2-30 carbon atoms, which may be hydroxylated and which are liquid at 20° C. These include hexyldecyl stearate, hexyldecyl laurate, isodecyl neopentanoate, isononyl isononanoate, C12-15 alkyl benzoate, 2-ethylhexyl palmitate, 2-ethylhexyl stearate, isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid-2-butyl octanoate, diisotridecyl acetate, n-butyl stearate, n-hexyl laurate, n-decyl oleate, oleyl oleate, oleyl erucate, erucyl oleate, erucyl erucate, ethylene glycol dioleate and dipalmitate.

Further preferred oils d) which can be used are chosen from the addition products of at least 6 ethylene oxide and/or propylene oxide units onto mono- or polyhydric C3-22-alkanols. Especially preferred are PPG-14 butyl ether and PPG-15 stearyl ether.

A particular preferred embodiment of the invention is characterized in that the emulsion comprises at least one oil selected from the addition products of 10 to 20 propylene oxide units onto mono- or polyhydric C3-22-alkanols, which are liquid at 20° C. Within this embodiment it is preferred if the oil d) is selected from PPG-14 butyl ether and PPG-15 stearyl ether.

In another embodiment of the invention the emulsion comprises at least one oil selected from the addition products of 10 to 20 propylene oxide units onto mono- or polyhydric C3-22-alkanols, which are liquid at 20° C., and/or esters of the linear or branched saturated or unsaturated fatty alcohols having 2-30 carbon atoms with linear or branched saturated or unsaturated fatty acids having 2-30 carbon atoms, which are liquid at 20° C. It is particular preferred if the oil d) is selected from PPG-14 butyl ether, PPG-15 stearyl ether and/or esters of the linear or branched saturated or unsaturated fatty alcohols having 2-30 carbon atoms with linear or branched saturated or unsaturated fatty acids having 2-30 carbon atoms, which are liquid at 20° C. It is even more preferred if PPG-14 butyl ether, PPG-15 stearyl ether and/or C12-15 alkyl benzoate are contained.

It is further preferred according to the invention if oils that are liquid at 20° C. are contained in a total quantity in the range from 10 to 30% by weight, more preferably 15 to 27% by weight and most preferably 18 to 25% by weight, calculated to the total weight of the emulsion.

Accordingly it is also preferred if PPG-14 butyl ether, PPG-15 stearyl ether and/or C12-15 alkyl benzoate are contained in a total quantity in the range from 10 to 30% by weight, more preferably 15 to 27% by weight and most preferably 18 to 25% by weight, calculated to the total weight of the emulsion. Thereby it is further preferred if at least PPG-14 butyl ether and C12-15 alkyl benzoate are contained. If PPG-14 butyl ether and C12-15 alkyl benzoate are contained the ratio by weight between PPG-14 butyl ether and C12-15 alkyl benzoate is preferably in the range from 10:1 to 1:1, more preferably 6:1 to 1.5:1, and most preferably 3:1 to 2:1.

According to the invention the emulsion comprises at least one nonionic emulsifier with an HLB value of more than 7. These are emulsifiers generally known to the person skilled in the art, as listed, for example, in Kirk-Othmer, “Encyclopedia of Chemical Technology”, 3rd edition, 1979, volume 8, page 913-916.

It is preferred according to the invention if the total quantity of the nonionic emulsifier with an HLB value of more than 7 is in the range from 0.5 to 3% by weight, more preferably 1 to 2.5% by weight, based on the total weight of the emulsion.

Particular preference is given to emulsifier e), which are C12-C18-alkanols having in each case 10-30 units of ethylene oxide per molecule, and mixtures of these substances. In particular preferred are ceteth-12, ceteth-20, ceteth-30, steareth-12, steareth-20, steareth- 21, oleth-20, steareth-30, laureth-12 and beheneth-20. It is most preferred if at least Steareth-21 and/or Oleth-20 are contained as emulsifier having an HLB value of more than 7. Accordingly it is also preferred if the emulsion comprises steareth-21 and/or oleth-20 in a total quantity from 0.5 to 3% by weight, more preferably 1 to 2.5% by weight, based on the total weight of the emulsion.

In other embodiments it is preferred if the at least one emulsifier e) is characterized in that it has an HLB value in the range from 14 to 17, more preferably from 14.5 to 16.5 and most preferably from 15 to 16. Accordingly it is also preferred if the emulsion comprises at least one emulsifier e) having an HLB value in the range from 14 to 17, more preferably from 14.5 to 16.5 and most preferably from 15 to 16, in a total quantity from 0.5 to 3% by weight, more preferably 1 to 2.5% by weight, based on the total weight of the emulsion.

Furthermore, the emulsion comprises at least one nonionic emulsifier with an HLB value greater than 1.0 and less than or equal to 7.0. It is preferred according to the invention if the nonionic emulsifier f) has an HLB value in the range from 3 to 6.5, more preferably 4 to 6. Those emulsifiers having an HLB value greater than 1.0 and less than or equal to 7.0, preferably from 3 to 6.5 and most preferably 4 to 6 are preferably contained in the emulsion in a total quantity from 0.1% to 1.5% by weight, more preferably 0.3 to 1% by weight, calculated to the total weight of the emulsion.

Preferred embodiments of the invention are characterized in that at least one nonionic emulsifier having a HLB value in the range from 3 to 6.5 is contained in a total quantity from 0.1% to 1.5% by weight, more preferably 0.3 to 1% by weight, calculated to the total weight of the emulsion.

Further preferred embodiments of the invention are characterized in that at least one nonionic emulsifier having a HLB value in the range from 4 to 6 is contained in a total quantity from 0.1% to 1.5% by weight, more preferably 0.3 to 1% by weight, calculated to the total weight of the emulsion.

A particular preferred nonionic emulsifier having an HLB value of 4.9 is Steareth-2. Accordingly it is preferred if the emulsion comprises at least Steareth-2 as nonionic emulsifier f). The quantity of Steareth-2 is preferably in the range from 0.1% to 1.5% by weight, more preferably 0.3 to 1% by weight, calculated to the total weight of the emulsion.

According to the invention it is further preferred if the emulsion comprises propylene glycol, whereby it is preferred if the total quantity of propylene glycol is in the range from 0.5 to 4% by weight calculated to the total weight of the emulsion.

Furthermore, it is preferred according to the invention, if the emulsion further comprises at least one fatty alcohol selected from the group consisting of cetyl alcohol and stearyl alcohol. Accordingly it is according to the invention if cetyl alcohol and/or stearyl alcohol are included. Mixtures of both ingredients are well known under the CTFA designation cetearyl alcohol.

In the case the emulsion comprises cetyl alcohol, stearyl alcohol and/or mixtures thereof it is preferred, if the total quantity of those alcohols is in the range from 1 to 5% and more preferably from 1.5% to 3.5% by weight calculated to the total weight of the emulsion.

According to the invention cetyl alcohol and/or stearyl alcohol are not considered as emulsifier e) or f) and also not as an oil or wax component c).

The water content of the emulsion is preferably in the range from 32 to 45% by weight calculated to the total weight of the emulsion.

Furthermore, the emulsion of the invention is preferably characterized in that that the emulsion is liquid at 70° C. and is solid under normal conditions.

After storing 24 hours under normal conditions the emulsion is preferably characterized in that it has a penetration force value in the range from 150-800 gram-force (g-force), preferably 200-750 gram-force (g-force), particularly preferably 350-600 gram-force (g-force), at a penetration depth of 5,000 mm. The penetration force value is a measure of the hardness of a stick (and also of a solid cream composition) and indicates at what maximum force a defined measuring probe, here a stainless steel 45° cone (model TA 15) is inserted perpendicularly (axially) into the antiperspirant mass to be measured at a feed rate of 2 mm/second to a penetration depth suitable for the measurement, here to a penetration depth of 5,000 mm. The determination of the penetration force value is carried out using the TA-XT2i texture analyzer from Stable Micro Systems (Vienna Court, Lammas Road, Godalming, Surrey GU7 1YL). The maximum force is given in gram-force (g-force). Here, lower values indicate a softer composition, harder compositions have a higher penetration force value. The measurements are carried out under normal conditions.

The present invention further provides a process for the preparation of an antiperspirant stick, wherein the wax and oil components c) and d) are heated together with the emulsifier e) and f) and optionally all further oil soluble ingredients to 90-95° C. and melted. Then the water containing the water-soluble ingredients is likewise heated to 90-95° C. is added with vigorous stirring, optionally further ingredients are mixed in. Afterwards the formed emulsion is stored at 70° C. and poured into suitable dispenser molds and solidified by static cooling (without further stirring) to normal conditions.

Furthermore, it is preferred if the emulsion of the invention does not comprise any particles which are insoluble in water and which are solid at any temperature above 95° C.

### EXAMPLES

The following examples should illustrate the compositions of this invention, without intending to limit the invention to these examples. The numerical values in the examples are percentages by weight, based on the total weight of the preparations.

| Ingredient | Com. 1 | Ex. 1 |
|---|---|---|
| Aluminum | 15 | 15 |
| Chlorohydrate | | |
| Palmitamidopropyltrimonium | — | 0.6 |
| Chloride | | |
| Cetyl Palmitate | 6 | 6 |
| Hydrogenated Castor | 6 | 6 |
| Oil | | |
| PPG-14 Butyl Ether | 15 | 15 |
| C12-15 Alkyl | 5.6 | 5.6 |
| Benzoate | | |
| C20-40 Alkyl Stearate | 1 | 1 |
| Steareth-21 | 0.5 | 0.5 |
| Steareth-2 | 0.5 | 0.5 |
| Oleth-20 | 1.0 | 1.0 |
| Fatty Acid mixture | 1.2 | 1.2 |
| consisting of 47% | | |
| Stearic Acid, 48.5% | | |
| Palmitic Acid, 2.5% | | |
| Myristic Acid, 1.5% | | |
| Arachidic Acid and | | |
| 0.5% Oleic Acid | | |
| Propylene Glycol | 2 | 2.4 |
| Cetearyl Alcohol | 2.5 | 2.5 |
| Parfum | q.s. | q.s. |
| Aqua | ad | ad |
| | 100 | 100 |
| Stability of the | Lumps | No |
| emulsion after storing | suspended | lumps |
| for 4 hours at 70° C. | in the | |
| | emulsion | |

The examples above were prepared in the following manner:
a) Heat the oil phase including cetyl palmitate, hydrogenated castor oil, palmitamidopropyltrimonium chloride, PPG-14 butyl ether and C12-15 alkyl benzoate, steareth-21, steareth-2, oleth-20, palmitic acid+stearic acid+myristic acid+arachidic acid+oleic acid, cetearyl alcohol or cetyl alcohol and stearyl alcohol and C20-40 alkyl stearate up to 90° C.
b) Heat the water phase including aluminum chlorohydrate, propylene glycol and water up to 85° C.
c) Bring both phases together under homogenizing to build the emulsion and then cool down to 70° C. and add the perfume and homogenize a second time.

Afterwards the obtained emulsions were stored at 70° C. for at least 4 hours. After 4 hours the emulsions were analyzed for lumps. Significant amounts of lumps were found in the comparative example Com. 1. The samples according to the invention Ex. 1 did not show any lumps after 4 hours storing at 70° C. Ex. 1 did also not show lumps after 8 hours storing at 70° C.

Further example formulations:

| Ingredient | Ex. 2 | Ex. 3 | Ex. 4 | Ex. 5 | Ex. 6 | Ex. 7 |
|---|---|---|---|---|---|---|
| Aluminum Chlorohydrate | 15 | 0 | 17 | 17 | 0 | 17 |
| Aluminum | 0 | 15 | 0 | 0 | 15 | 0 |
| Sesquichlorohydrate | | | | | | |
| Palmitamidopropyl- | 0.8 | 0.5 | 1 | 0.6 | 1.2 | 1.2 |
| trimonium Chloride | | | | | | |
| Cetyl Palmitate | 7 | 6 | 5 | 6 | 6 | 5 |
| PPG-14 Butyl Ether | 18 | 15 | 16 | 15 | 15 | 15 |
| Hydrogenated Castor Oil | 5 | 6 | 7 | 6 | 6 | 6 |
| C12-15 Alkyl Benzoate | 7 | 5.6 | 6 | 5.6 | 5.6 | 5.6 |
| C20-40 Alkyl Stearate | 0.9 | 0.9 | 1.1 | 1 | 1.1 | 1 |
| Steareth-21 | 0.55 | 0.6 | 0.5 | 0.5 | 0.4 | 0.5 |
| Steareth-2 | 0.55 | 0.6 | 0.5 | 0.5 | 0.6 | 0.5 |
| Oleth-20 | 1.1 | 0.9 | 1 | 0.8 | 1 | 1.0 |
| Fatty Acid mixture | 4 | 3.5 | 3.8 | 3.8 | 4 | 3.8 |
| consisting of 47% Stearic | | | | | | |
| Acid, 48.5% Palmitic Acid, | | | | | | |
| 2.5% Myristic Acid, 1.5% | | | | | | |
| Arachidic Acid and 0.5% | | | | | | |
| Oleic Acid | | | | | | |
| Parfume | q.s | q.s | q.s | q.s | q.s | q.s |
| Propylene Glycol | 2.0 | 2.2 | 1.5 | 2.0 | 1.0 | 2.0 |
| Cetearyl Alcohol | 3 | 2.5 | 3 | 2.5 | 2.8 | 2.5 |
| Aqua | Ad. | Ad. | Ad. | Ad. | Ad. | Ad. |
| | 100 | 100 | 100 | 100 | 100 | 100 |

1-24. (canceled)

25. An antiperspirant oil-in-water emulsion, wherein the emulsion comprises:
(a) at least one antiperspirant active aluminum compound;
(b) palmitamidopropyl trimonium chloride;
(c) at least one lipid and/or wax component having a melting point of >50° C.
(d) at least one oil which is liquid at 20° C.;
(e) at least one nonionic emulsifier having an HLB value of higher than 7;
(f) at least one nonionic emulsifier having an HLB value of higher than 1.0 and lower than or equal to 7.0; and
(g) from 30% to 50% by weight of water, based on a total weight of the emulsion.

26. The emulsion of claim 25, wherein (a) comprises one or more of aluminum chlorohydrate; aluminum sesquichlorohydrate; aluminum zirconium chlorohydrate and a aluminum zirconium chlorohydrate glycine complex.

27. The emulsion of claim 25, wherein the emulsion comprises from 5% to 30% by weight of (a), based on a total weight of the emulsion.

28. The emulsion of claim 25, wherein (a) consists of aluminum chlorohydrate.

29. The emulsion of claim 25, wherein the emulsion comprises from 0.1% to 5% by weight of (b), based on a total weight of the emulsion.

30. The emulsion of claim 25, wherein the emulsion comprises from 8% to 18% by weight of (c) based on a total weight of the emulsion.

31. The emulsion of claim 25, wherein the wax component (c) comprises at least one ester of a saturated monohydric C16-C60-alcohol and a saturated C8-C36-monocarboxylic acid.

32. The emulsion of claim 25, wherein the wax component (c) comprises one or more esters of a saturated, branched or unbranched alkanecarboxylic acid having a chain length of from 12 to 24 carbon atoms and a saturated, branched or unbranched alcohol having a chain length of from 16 to 50 carbon atoms.

33. The emulsion of claim 32, wherein the one or more esters of a saturated, branched or unbranched alkanecarboxylic acid having a chain length of from 12 to 24 carbon atoms and a saturated, branched or unbranched alcohol having a chain length of from 16 to 50 carbon atoms are present in a concentration of from 3% to 9% by weight, based on a total weight of the emulsion.

34. The emulsion of claim 25, wherein (c) comprises a C20-C40-alkyl stearate.

35. The emulsion of claim 25, wherein (c) comprises hydrogenated castor oil,

36. The emulsion of claim 25, wherein (c) comprises one or more saturated linear C14-C36-carboxylic acids as wax component.

37. The emulsion of claim 25, wherein (c)comprises a C20-C40-alkyl stearate.

38. The emulsion of claim 25, wherein (d) comprises at least one addition product of at least 6 ethylene oxide and/or propylene oxide units onto a mono- or polyhydric C3-22-alkanol.

39. The emulsion of claim 25, wherein (d) comprises one or more of PPG-14 butyl ether. PPG-15 stearyl ether and C12-15 alkyl benzoate

40. The emulsion of claim 25, wherein the emulsion comprises from 0.5% to 3% by weight of (e), based on a total weight of the emulsion.

41. The emulsion of claim 25, wherein(e) comprises one or more C12-C18-alkanols comprising 10-30 units of ethylene oxide per molecule.

42. The emulsion of claim 25, wherein the emulsion comprises from 0.1% to 1.5% by weight of (f), based on a total weight of the emulsion,

43. The emulsion of claim 25, wherein (f) comprises steareth-2.

44. The emulsion of claim 25, wherein the emulsion is liquid at 70° C. and solid at room temperature.
